(19) United States
(12) Patent Application Publication
(10) Pub. No.: US 2006/0122165 A1
(43) Pub. Date: Jun. 8, 2006

(54) Crystalline cefdinir

(76) Inventors: Otto Daemon, Jenbach (AT); Klaus Hartmann, Kufstein (AT); Johannes Raneburger, Worgl (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/294,116

(22) Filed: Dec. 5, 2005

(30) Foreign Application Priority Data
Dec. 7, 2004 (GB) GB0426837.1

(51) Int. Cl. A61K 31/545; C07D 501/14

(52) U.S. Cl. 514/202; 540/222

(57) ABSTRACT

The present invention relates to a new crystalline form of cefdinir and processes for the preparation thereof. Furthermore, the present invention relates to pharmaceutical compositions comprising said new crystalline form of cefdinir and to processes for preparing these compositions.

### FIELD OF THE INVENTION

The present invention relates to cefdinir. More particularly to a new crystalline form of cefdinir and processes for the preparation thereof. Furthermore, the present invention relates to pharmaceutical compositions comprising said new crystalline form of cefdinir and to processes for preparing them.

### BACKGROUND OF THE INVENTION

Cefdinir, 7-[2-(2-aminothiazol-4-yl)-2-hydroxyiminoacetamido]-3-vinyl-3-cephem-4-carboxylic acid, a cephalosporin known to have high antibiotic activity, is described in U.S. Pat. No. 4,559,334. A crystalline form of cefdinir, identified as form A, is claimed in U.S. Pat. No. 4,935,507. Briefly, crystalline cefdinir is described to be obtainable by crystallization from acidified solutions at ambient temperatures (see e.g. Example 2 in column 12). Form A is comprised in the commercially available form of cefdinir, e.g. as sold under the trademark Omnicef®. US 2003/0204082 describes a further crystalline form of cefdinir which is herein referred to as crystalline form B. Briefly, crystalline form B of cefdinir is described to be obtainable from aqueous solutions by acidification after cooling (see Example 1 on page 1).

Different crystal forms of one compound may interconvert, that is under certain conditions one crystal form with favourable characteristics may convert to another crystal form with possibly less favourable characteristics. There is thus a need for a pharmaceutical composition comprising cefdinir in a defined polymorphic state.

### SUMMARY OF THE INVENTION

In one aspect, the present invention provides a novel cefdinir crystal modification, hereinafter designated as form C. The new crystal form C according to the invention is found to be a trihydrate form of cefdinir, and is distinguished from previously known forms of cefdinir by physical and spectroscopic properties, for instance, x-ray powder diffraction pattern, or raman spectrum.

The novel crystalline form C of cefdinir is stable at environmental conditions having a relative humidity of at least about 45%.

The crystalline form C of the present invention may be advantageously prepared in an environmentally friendly manner by crystallization from aqueous solution.

Additionally, the present invention provides a process for the preparation of this new crystalline form C.

In another aspect the present invention provides a pharmaceutical composition comprising crystalline form C of cefdinir, wherein the equilibrium relative humidity of said composition is at least about 45%, preferably at least about 48% as measured by the ERH method as herein described.

In an additional aspect, the present invention provides a container comprising a pharmaceutical composition comprising crystalline form C of cefdinir and a gaseous atmosphere having a relative humidity of at least about 45%, preferably of at least about 48%.

Additionally, the present invention provides processes for preparing a pharmaceutical composition comprising crystalline form C of cefdinir.

Furthermore, the present invention provides crystalline form C of cefdinir for use as a medicament.

The pharmaceutical composition of the invention as prepared according to the processes of the invention stabilizes cefdinir in its novel crystalline form C over a period of at least 6 months by the use of a container being capable of maintaining a gaseous atmosphere having a relative humidity of at least about 45%.

### BRIEF DESCRIPTION OF THE DRAWINGS

FIG. 1 shows the powder x-ray diffraction pattern of the form C crystal modification of cefdinir (CuK_{α} radiation source, voltage 40 KV, tube current 35 mA, scan rate 0.005° 2θ s⁻¹, angular range 2 to 40° 2θ)

FIG. 2 shows the Raman Spectra of the form C crystal modification of cefdinir (Bruker RFS 100 Raman spectrometer, Nd:YAG Laser 1064 nm excitation source, Ge detector, resolution 4 cm⁻¹).

### DETAILED DESCRIPTION OF THE INVENTION

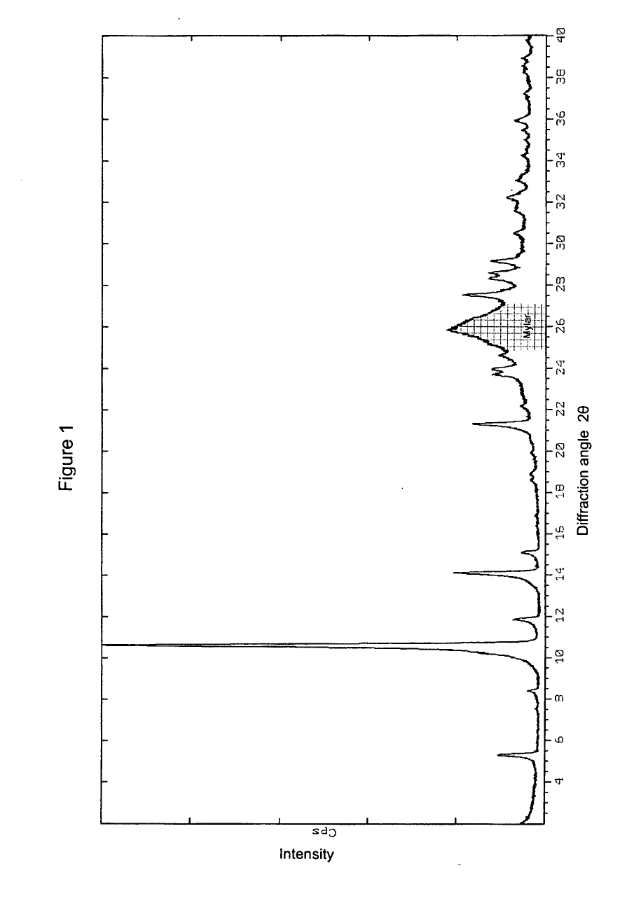

The powder x-ray diffraction pattern of the form C crystal modification of cefdinir according to the present invention shows characteristic peaks at the d-spacings of about 16.68, 8.33, 6.28, 4.17 and 3.24. The powder x-ray diffraction pattern for the form C crystal modification of cefdinir depicted in FIG. 1 is summarized in Table 1:

Table 1:

TABLE 1
Powder x-ray diffraction data for cefdinir form C polymorph

| ° 2 theta | d (Å) | Relative Intensity (%) |
|---|---|---|
| 5.3 | 16.68 | 11 |
| 8.4 | 10.54 | 4 |
| 10.6 | 8.33 | 100 |
| 14.1 | 6.28 | 21 |
| 15.1 | 5.86 | 5 |
| 21.3 | 4.17 | 16 |
| 23.7 | 3.75 | 12 |
| 24.0 | 3.71 | 12 |
| 24.6 | 3.61 | 11 |
| 26.3 | 3.38 | 17 |
| 27.5 | 3.24 | 19 |
| 28.3 | 3.15 | 13 |
| 28.6 | 3.12 | 13 |
| 29.2 | 3.06 | 12 |
| 30.5 | 2.93 | 7 |
| 31.6 | 2.83 | 7 |
| 32.2 | 2.77 | 9 |
| 33.0 | 2.71 | 7 |
| 35.9 | 2.50 | 7 |

The form C crystal modification of cefdinir according to the present invention may be described as crystalline 7-[2-(2-aminothiazol-4-yl)-2-hydroxyiminoacetamido]-3-vinyl-3-cephem-4-carboxylic acid having powder x-ray diffraction peaks at d-spacings of about 16.68, 8.33, 6.28, 4.17 and 3.24. The form C crystal modification of cefdinir according to the present invention may further present peaks at any one or more d-spacing selected from the following d-spacings: about 3.75, 3.71, 3.61, 3.38, 3.15, 3.12 and 3.06.

The form C crystal modification of cefdinir according to the present invention may also be described as crystalline 7-[2-(2-aminothiazol-4-yl)-2-hydroxyiminoacetamido]-3-vinyl-3-cephem-4-carboxylic acid having the powder x-ray diffraction pattern depicted in Table 1.

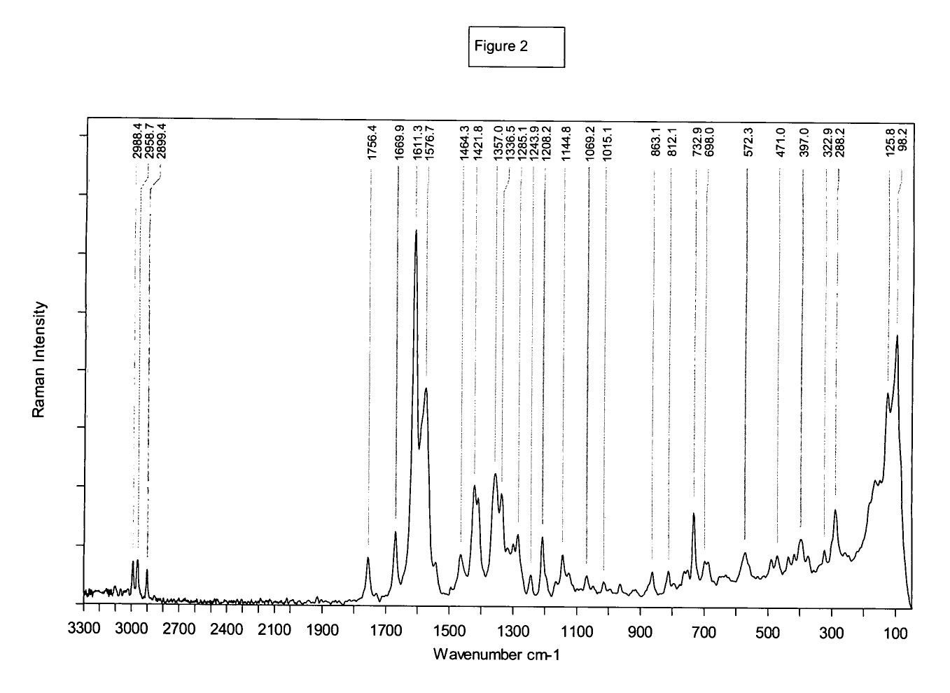

Form C crystal modification of cefdinir according to the present invention may also be properly described as crystalline 7-[2-(2-aminothiazol-4-yl)-2-hydroxyiminoacetamido]-3-vinyl-3-cephem-4-carboxylic acid having the Raman spectrum depicted in FIG. 2.

The crystalline form C of cefdinir according to the present invention has been shown to be a trihydrate form. Under thermogravimetric analysis (TGA-7) the C crystal form of the present invention is shown to be a hydrated form with a water content of about 12% (w/w) which corresponds to the stoichiometry of a trihydrate.

Accordingly the present invention provides crystalline 7-[2-(2-aminothiazol-4-yl)-2-hydroxyiminoacetamido]-3-vinyl-3-cephem-4-carboxylic acid trihydrate. The form C crystal modification of cefdinir according to the present invention may accordingly also be described as crystalline 7-[2-(2-aminothiazol-4-yl)-2-hydroxyiminoacetamido]-3-vinyl-3-cephem-4-carboxylic acid trihydrate having powder x-ray diffraction peaks at d-spacings of about 16.68, 8.33, 6.28, 4.17 and 3.24, or the powder x-ray diffraction pattern depicted in Table 1. Form C crystal modification of cefdinir according to the present invention may accordingly also be described as crystalline 7-[2-(2-aminothiazol-4-yl)-2-hydroxyiminoacetamido]-3-vinyl-3-cephem-4-carboxylic acid trihydrate having the Raman spectrum depicted in FIG. 2.

According to one embodiment the crystalline form of cefdinir according to the present invention may be prepared by crystallizing from a solution of 7-[2-(2-aminothiazol-4-yl)-2-hydroxyiminoacetamido]-3-vinyl-3-cephem-4-carboxylic acid, adjusted to pH of 1.5 to 4 at a temperature of from −5° C. to 6° C., preferably from 0° C. to 5° C. Followed by drying the thus obtained crystals at a relative humidity (RH) of at least 40%, preferably at least 50%.

The crystals are preferably dried to reach an equilibrium relative humidity of at least 50%, preferably at least 60%, more preferably at least 70%.

Advantageously the new crystalline form C of cefdinir according to the present invention may be prepared by crystallization from an aqueous solution. Preferably the form C crystal modification of cefdinir may be crystallized from a solution in water.

Alternatively the solvent system for the crystallization comprises a mixture of water and at least one organic solvent. Suitable organic solvents include all water miscible organic solvents including alcohols such as methonal or ethanol, ketones such as acetone, ethers such as tetrahydrofuran, carboxcylic acids and carbocylic acid derivatives such as acetic acid, dimethylacetamide, dimethylformamide, ethyl acetate, and sulfoxides such as DMSO or any combination thereof.

The pH of the solution should be adjusted to pH 1.5 to 4, preferably the pH is maintained in the range of pH 2 to 3. The pH may adjusted by conventional methods such as by the addition of a dilute inorganic or organic acid, exemplary acids include dilute hydrochloric acid or sulphuric acid.

According to one embodiment drying of the crystals may be carried out by drying under a constant vapour/gas stream, such as a continuous air or nitrogen stream. The relative humidity of the vapour/gas stream may be adjusted to provide the desired relative humidity for drying of at least 40%. The crystals may be dried until they reach the desired equilibrium relative humidity of at least 50%. Other methods suitable for drying the crystals will be evident to the skilled person.

The starting solution of 7-[2-(2-aminothiazol-4-yl)-2-hydroxyiminoacetamido]-3-vinyl-3-cephem-4-carboxylic acid for the crystallization of the crystal form C of cefdinir according to the present invention may be prepared by dissolution, e.g. in water, or in a mixture of water and one or more organic solvents, of the compound cefdinir obtained by known methods, for instance as disclosed in U.S. Pat. No. 4,559,334.

Alternatively the solution of 7-[2-(2-aminothiazol-4-yl)-2-hydroxyiminoacetamido]-3-vinyl-3-cephem-4-carboxylic acid for the crystallization of the crystal form C of cefdinir may be obtained from a known intermediate compound in the synthesis of cefdinir. Suitable intermediate compounds include salts and solvates of acetyl cefdinir and trityl cefdinir, for example acid addition salts of acetyl cefdinir, including sulfate, methane sulphonate, benzene sulphonate, toluene sulphonate, phosphate, hydrogen chloride salts.

According to one embodiment of the present invention the solution of 7-[2-(2-aminothiazol-4-yl)-2-hydroxyiminoacetamido]-3-vinyl-3-cephem-4-carboxylic acid for the crystallization of the crystal form C of cefdinir may be prepared from a salt of acetyl cefdinir, e.g. according to the method described in WO 2004/701662.

In another aspect of the invention, crystalline form C of cefdinir can be obtained by conversion of the crystalline form B of cefdinir, obtainable as described in US 2003/0204082 in Example 1, into crystalline form C of cefdinir. Cefdinir form B is in the form of a hydrate. Said conversion takes place within minutes upon exposure of pure crystalline form B to a relative humidity of at least about 65%. Alternatively, conversion of the crystalline form B of cefdinir into crystalline form C of cefdinir may be performed by simply exposing pure form B crystals of cefdinir to an aqueous suspending medium, preferably to water, where conversion takes place within seconds.

The crystalline form of cefdinir according to the present invention may be used for the preparation of pharmaceutical compositions of cefdinir.

The pharmaceutical compositions of the invention comprise crystalline form C of cefdinir and preferably have an equilibrium relative humidity of at least about 45%, e.g. of at least 45%, more preferably of at least about 48%, e.g. of at least 48%, e.g. of at least 50%, as measured by the herein described ERH method. The pharmaceutical compositions of the invention may also have an equilibrium relative humidity of at least about 65% or of at least 90% or more, e.g. may be in a liquid form, e.g. in the form of a liquid suspension.

More preferably, the pharmaceutical compositions of the invention comprising crystalline form C of cefdinir have the above mentioned equilibrium relative humidity at a temperature of from about 20° C. to about 30°, e.g. about room temperature, more preferably at a temperature of about 25±1° C.

The equilibrium relative humidity of the pharmaceutical compositions or of crystalline form C of cefdinir as herein described is measured by determining the relative humidity in % in the air above a test sample, e.g. a pharmaceutical composition of the invention, after establishment of a humidity equilibrium in a closed system at a constant temperature according to the following method: the equipment used is the commercially available measuring chamber Rotronic AW-VC comprising a hygrometer of the type BT-RS1. The test sample, e.g. a pharmaceutical composition of the invention, is filled into a sampling dish which is placed into the measuring chamber which has been thermostated to a temperature of 25±1° C., said chamber is subsequently closed and sealed. After establishment of an equilibrium of the relative humidity which state is typically shown by the disappearance of a trend indication, the value of the relative humidity in % is read from the hygrometer. Relative humidity is defined as the equilibrium relative humidity of the pharmaceutical compositions as measured as herein described. Filling of the chamber is performed according to the instructions of the manufacturer. In case the test sample is a powder or granules for oral suspension, or a liquid suspension, said sample is directly placed into the above mentioned sampling dish. In case the test sample is a capsule, the appropriate number of capsules is opened and their contents is filled into the sampling dish. In case the test sample is a tablet, the appropriate number of tablets is crushed by using a mortar, and filled into the sampling dish. The above described preparation of the test samples before measurement is to be performed at room temperature, i.e. at a temperature of 20° C. to 30° C., such as at about 25° C., and at a relative humidity of 45% to 60%. Furthermore said preparation is to be performed as quickly as possible and uninterrupted within a maximum of 30 minutes. The above described method for measurement of the equilibrium relative humidity of the pharmaceutical compositions of the invention is herein also called ERH method.

The pharmaceutical compositions of the invention may further comprise one or more pharmaceutically acceptable excipients which are preferably selected from the group consisting of fillers, sweeteners, buffering agents, glidants, flowing agents, fllavouring agents, lubricants, preservatives, surfactants, wetting agents, binders, disintegrants and thickeners. Other excipients known in the field of pharmaceutical compositions may also be used. Furthermore, the pharmaceutical compositions may comprise a combination of 2 or more excipients also within one of the members of the above mentioned group. Preferably, the fillers are also sweeteners.

Preferred fillers are sucrose, mannitol and cellulose. Preferred sweeteners are sucrose and Aspartame. Preferred buffering agents are citric acid and sodium citrate. A preferred glidant or flowing agent is colloidal silica, e.g. Aerosil®. Preferred flavours are strawberry and raspberry. A preferred lubricant is magnesium stearate. A preferred preservative is sodium benzoate. A preferred surfactant and/or wetting agent is sodium lauryl sulfate. Preferred disintegrants are cross-linked polyvinylpyrrolidones, e.g. crospovidones, cross-linked sodium carboxymethylcellulose, e.g. croscarmellose sodium, and calcium-carmellose. Preferred thickeners are xanthan gum and guar gum. Examples of suitable binders include starches and modified starches, e.g., pregelatinized starch, celluloses, e.g. hydroxypropylmethylcellulose, hydroxypropyl cellulose, hydroxyethyl cellulose and polyvinyl pyrrolidone, e.g., Povidone.

Optionally, the pharmaceutical compositions of the invention may comprise a coating which comprises a film-forming agent such as e.g. cellulose and derivatives thereof, hydroxypropylmethylcellulose, polyvinylpyrrolidone, or enteric film-forming agents such as cellulose phthalates, poly(meth)acrylates and polyvinyl acetate-phthalate.

The pharmaceutical compositions of the invention are preferably forms for oral administration, and may be in the form of a tablet, a capsule, a caplet, granules for oral suspension, a powder for oral suspension, a dispersible tablet, or in the form of a liquid suspension.

Preferably, the pharmaceutical compositions of the invention comprise crystalline form C of cefdinir, and preferably further comprise one or more pharmaceutically acceptable excipients selected from the group consisting of a filler, a sweetener, a thickener, a flowing agent, a flavour, a lubricant, a surfactant and a disintegrant. The pharmaceutical compositions of the invention may further comprise one or more pharmaceutically acceptable excipients selected from the group consisting of buffering agents, binders, glidants, preservatives and wetting agents.

The pharmaceutical compositions of the invention may additionally comprise a suspending medium, preferably an aqueous suspending medium, more preferably water.

In a further embodiment of the invention, the pharmaceutical composition comprising crystalline form C of cefdinir may be in the form of a liquid suspension obtained by a process comprising steps a″) to d″) as described below.

The pharmaceutical compositions of the invention may be prepared according to the following process comprising the steps of:

a′) equilibrating a crystalline form of 7-[2-(2-aminothiazol-4-yl)-2-hydroxyiminoacetamido]-3-vinyl-3-cephem-4-carboxylic acid in the form of a hydrate at a relative humidity of at least about 63%, preferably of at least 65%,

b′) mixing the equilibrated 7-[2-(2-aminothiazol-4-yl)-2-hydroxyiminoacetamido]-3-vinyl-3-cephem-4-carboxylic acid obtained in step a) with one or more pharmaceutically acceptable excipients at a relative humidity of at least about 45%,

c′) optionally granulating the mixture obtained in step b′) at a relative humidity of at least about 45%, and

d′) further processing the mixture obtained in step b′) or the granulate obtained in step c′) at a relative humidity of at least about 45% to obtain a pharmaceutical composition.

The equilibrating procedure of step a′) is preferably performed at a relative humidity of about 63% to about 75% , more preferably of about 65% to about 70%, such as of at 65% to 70%. Said equilibration may be performed in a gaseous atmosphere being e.g. air or nitrogen, preferably air having the above mentioned relative humidity. Above about 63% relative humidity, for example cefdinir form B crystals obtainable as described above convert to form C crystals. Form C crystals—once formed—are stable above a relative humidity of at least 45%.

The equilibration in step a′) may be performed in a suitable way by exposing a crystalline form of 7-[2-(2-aminothiazol-4-yl)-2-hydroxyiminoacetamido]-3-vinyl-3-cephem-4-carboxylic acid in the form of a hydrate to a gaseous atmosphere, e.g. to air or nitrogen, preferably to air, being adjusted to the relative humidity as described above. The process of equilibration is preferably continued until cefdinir is transformed to only form C crystals.

The equilibration may be performed by flushing a crystalline form of 7-[2-(2-aminothiazol-4-yl)-2-hydroxyiminoacetamido]-3-vinyl-3-cephem-4-carboxylic acid in the form of a hydrate with a gaseous atmosphere, e.g. with air or nitrogen, preferably with air, being adjusted to a relative humidity of at least about 63%, preferably of about 65% to about 95% , more preferably of about 70% to about 90%, such as of 70% to 90%.

Alternatively, the equilibration may be performed flushing the crystalline form of 7-[2-(2-aminothiazol-4-yl)-2-hydroxyiminoacetamido]-3-vinyl-3-cephem-4-carboxylic acid in the form of a hydrate with a gaseous atmosphere, e.g. with air or nitrogen, preferably with air, being adjusted to the relative humidity as described above in a fluidized bed dryer or in a perforated drum or barrel. Other methods of performing said equilibration step are known.

Steps b′), c′) and d′) are preferably performed at a relative humidity of at least about 45%, e.g. of at least 45%, preferably of at least about 48%, such as of at least 48%, e.g. of at least about 50%, for example at a relative humidity of about 45% to about 70%. Said relative humidity is preferably the ambient relative humidity. The presence of the herein described relative humidity is believed to stabilize the crystalline form C of cefdinir its form throughout the herein mentioned process. The term “stabilize” as used herein relating to the crystalline form C is understood to mean that the novel form C of cefdinir is maintained in its crystalline trihydrate form, over a prolonged period of time as herein specified.

The optional granulation of step c′) may be performed according to, e.g. analogous to known methods such as dry or wet granulation, for example using a roller compactor or a fluidized bed granulator according, e.g. analogous to known methods.

The further processing of step d′) may comprise mixing the product obtained in step b′) or step c′) with additional pharmaceutically acceptable excipients, and optionally compressing the product obtained into tablets according to, e.g. analogously to known methods. Optionally, the excipients used in the above described process may be divided into portions wherein a first portion is used in step a′) and a further portion is used in steps b′), c′) or d′).

In another aspect, step d′) comprises the filling of the granulate obtained in step c′) into capsules, such as hard gelatine capsules.

The products or intermediate products obtained in the various steps of above described process are preferably stored at an ambient relative humidity of at least about 45%, e.g. of at least 45%, preferably of at least about 48%, e.g. of at least 48%, such as of at least about 50%, e.g. of at least about 55%. Said products may thus be stored in aluminium barrels or drums, in so-called Nirosta® drums, such as commercially available as Müller® drums. Said drums may be made gas-tight, e.g. air-tight by applying a sealing means, such as sealing rings to the lid thereof. Said products may also be stored in containers made of aluminium or Nirosta®-material as mentioned above whereof the closures or lids are provided with a sealing means, such as a sealing ring.

The pharmaceutical compositions obtained in step d′) are preferably packaged or filled into containers as herein described at an ambient relative humidity of at least about 45%, e.g. of at least 45%, preferably of at least about 48%, e.g. of at least 48%, such as at least about 50%, e.g. at least about 55%, for example at a relative humidity of about 45% to about 70%. Subsequently, said containers are tightly closed as herein described. Preferably, said containers are used for storage of the pharmaceutical compositions of the invention, for example at room temperature, such as at a temperature of about 20° C. to 30° C., e.g. at about 25° C., for a prolonged period, e.g. for at least 6 months, such as for at least 12 months, preferably for at least about 24 months, e.g. for up to 24 months, preferably for up to about 36 months, such as for up to about 60 months.

In a further aspect, the invention provides a container comprising a pharmaceutical composition according to the invention and a gaseous atmosphere having a relative humidity of at least about 45%, e.g. of at least 45%, preferably of at least about 48%, e.g. of at least 48%, such as of at least about 50%, e.g. of at least about 55%. Preferably, said containers are capable of maintaining a gaseous atmosphere having a relative humidity of at least about 45%, e.g. of at least 45%, preferably of at least about 48%, e.g. of at least 48%, such as of at least about 50%, e.g. of at least about 55%, for at least 6 months, such as for at least 12 months, preferably for at least 24 months, preferably for up to 36 months, such as for up to about 60 months. The gaseous atmosphere having a relative humidity of at least about 45%, e.g. of at least 45%, preferably of at least 48%, e.g. of at least 48%, such as of at least about 50%, e.g. of at least about 55%, is preferably air or nitrogen, more preferably air.

Said container is preferably a bottle, e.g. a glass or plastic bottle, e.g. a polyethylene bottles, such as known as securitainer, having e.g. a screw closure, or is a blister, e.g. an aluminium blister or strip, e.g. a blister consisting of 2 aluminium foils or strips, or a blister comprising an Aclar®) foil and an aluminium cover foil, or may be any other suitable container. More preferably said container is a gas-tight container, such as an air-tight container.

After the pharmaceutical compositions of the invention have been filled into the herein mentioned containers, said containers are preferably tightly closed, e.g. tightly or hermetically sealed, e.g. in a way to prevent any gaseous atmosphere from diffusing through the walls and/or closure of said containers. Methods of tightly sealing and/or closing said containers are known, such as sealing of glass or plastic bottles by applying an aluminium membrane to the bottle opening of said bottle by induction sealing and by applying a closure, e.g. a screw closure, or such as sealing of alu-alu blisters or strips, of blisters comprising an Aclar® foil and an aluminium cover foil by heat sealing according, e.g. analogous to known methods.

Preferred containers are glass or plastic bottles sealed with an aluminium membrane, alu-alu-blisters or strips, or blisters comprising an Aclar® foil and an aluminium cover foil. The container according to the invention is obtained by filling the pharmaceutical compositions of the invention into said container under the conditions as herein described.

Within said preferably tightly sealed container comprising a gaseous atmosphere, preferably air, a relative humidity of at least about 45%, e.g. of at least 45%, preferably of at least about 48%, e.g. of at least 48%, such as of at least about 50%, e.g. of at least about 55%, is maintained stable for at least 6 months, such as for at least 12 months, preferably for at least 24 months, preferably for up to 36 months, such as for up to about 60 months. Thereby, the crystalline form C of cefdinir comprised in the pharmaceutical compositions of the invention is stabilized in its trihydrate form as herein described over a period of up to at least 6 months, e.g. for at least 12 months, preferably for at least about 24 months, preferably for up to 36 months, such as for up to about 60 months.

Thus, the present invention also provides the use of a container capable of maintaining a gaseous atmosphere at a relative humidity of at least about 45%, e.g. of at least 45%, preferably of at least about 48%, e.g. of at least 48%, for up to at least 6 months, e.g. for at least 12 months, preferably at least 24 months for storage of a pharmaceutical composition of the invention. The present invention additionally provides the use of a gaseous atmosphere having a relative humidity of at least about 45%, e.g. of at least 45%, preferably of at least about 48%, e.g. of at least 48%, to stabilize the crystalline form C of cefdinir.

In a further aspect, the invention provides a pharmaceutical composition comprising crystalline form C of cefdinir being obtainable by the processes as herein described preferably under the conditions as herein described with regard to starting material, relative humidity and further processing. Furthermore, said pharmaceutical compositions may be packaged and/or stored under the conditions and for the time period as herein described.

The temperature applied during the herein described processes is preferably room temperature, e.g. is a temperature of about 20° C. to about 30° C., such as about 25° C.

The pharmaceutical compositions of the invention may comprise a therapeutically effective amount of crystalline form C of cefdinir. The pharmaceutical compositions of the invention may comprise for example units doses of about 1 mg to about 2000 mg of cefdinir.

Furthermore, the present invention provides cefdinir crystalline form C for use as a medicament, e.g. for the treatment of infectious diseases caused by germs which are sensitive to cefdinir. Said diseases are for example upper and lower respiratory tract infections and infections of skin and soft tissues.

In another aspect of the invention, cefdinir crystalline form C may be used for the preparation of a medicament for the treatment of the herein mentioned infectious diseases.

There is also provided a method of treating infectious diseases caused by germs sensitive to cefdinir by administering crystalline form C to a mammal, e.g. a human patient, in need thereof.

Additionally, the pharmaceutical compositions according to the invention may be used as a medicament and/or for the preparation of a medicament for the treatment of the herein mentioned infectious diseases, e.g. in humans.

The novel crystalline form C of cefdinir is advantageously stable at environmental conditions having a relative humidity of more than about 45%, e.g. of more than 45%, e.g. of more than about 48%, e.g. of at least about 50%, such as from 50% to 90%. Thus crystalline form C of cefdinir is suitable for easy handling during the manufacturing processes as used in the pharmaceutical industry, particularly in countries with a humid climate.

The pharmaceutical composition of the invention as manufactured according to the processes of the invention stabilizes cefdinir in its trihydrate form for a period of at least 6 months, e.g. of at least 12 months, preferably of at least 24 months, such as up to 36 months, e.g. for up to about 60 months by the use of a container according to the invention being capable of maintaining a gaseous atmosphere having a relative humidity of at least about 45%, e.g. of at least 45%, preferably of at least about 48%, e.g. of at least 48%, over the above mentioned period of time, said container being preferably closed, e.g. sealed tightly, preferably bearing a gas-tight closure or sealing.

Additionally, the pharmaceutical compositions comprising novel crystalline form C of cefdinir according to the present invention surprisingly exhibit favorable physico-chemical characteristics when compared to the form A described above. In particular at low pH the pharmaceutical compositions comprising novel crystalline form C of cefdinir according to the present invention surprisingly exhibit also favourable in vivo release characteristics in humans.

The present invention is further illustrated by the following non-limiting examples.

### EXAMPLE 1

### Preparation of form C from hydrochloride of (6R,7R)-7-[[(2Z)-(2-amino-4-thiazolyl)(acetoxyimino)acetyl]amino]-3-ethenyl-8-oxo-5-thia-1-azabicyclo[4.2.0]oct-2-ene-2-carboxylic acid

14.0 g hydrochloride of (6R,7R)-7-[[(2Z)-(2-amino-4-thiazolyl)(acetoxyimino)acetyl]amino]-3-ethenyl-8-oxo-5-thia-1-azabicyclo[4.2.0]oct-2-ene-2-carboxylic acid (Acetyl-Cefdinir Hydrochloride) are suspended at 0-5° C. in 64 ml methanol. 3.1 ml conc. sulphuric acid are added at this temperature. The solution is stirred for 2.5 hours and added dropwise into 450 ml of aqueous sodiumhydrogencarbonate solution. The pH of this solution is adjusted to 5.5 by sulphuric acid. 1.2 g activated carbon are then added and the solution is stirred for 30 min at 0-5° C. The activated carbon is then filtered off.

For crystallisation the filtrate is added dropwise into 280 ml water, and the pH is maintained between 2 and 3 by simultaneous addition of dilute sulphuric acid. After completion the crystal suspension is stirred for 1 h at 0-5° C. The white crystals are isolated by filtration and washed 3 times with 60 ml cold water each.

Drying of the crystals is done in a continuous air stream equilibrated to relative humidity (RH) of 52% at 25° C. until the crystals reach an equilibrium relative humidity of 58% at 25° C. (RH measurement carried out with a Vaisala HUMICAP® humidity and temperature meter HM70), to give (6R,7R)-7-[[(2Z)-(2-amino-4-thiazolyl)(acetoxyimino)hydroxy]amino]-3-ethenyl-8-oxo-5-thia-1-azabicyclo[4.2.0]oct-2-ene-2-carboxylic acid crystals (form C), 11.1 g.

### EXAMPLE 2

### Preparation of form C from hydrochloride of (6R,7R)-7-[[(2Z)-(2-amino-4-thiazolyl)(acetoxyimino)acetyl]amino]-3-ethenyl-8-oxo-5-thia-1-azabicyclo]4.2.0]oct-2-ene-2-carboxylic acid

14.0 g hydrochloride of (6R,7R)-7-[[(2Z)-(2-amino-4-thiazolyl)(acetoxyimino)acetyl]amino]-3-ethenyl-8-oxo-5-thia-1-azabicyclo[4.2.0]oct-2-ene-2-carboxylic acid (Acetyl-Cefdinir Hydrochloride) are suspended at 0-5° C. in 64 ml methanol. 3.1 ml conc. sulphuric acid are added at this temperature and the solution is stirred for 2.5 hours and added dropwise into 450 ml of aqueous sodiumhydrogencarbonate solution. The pH of this solution is adjusted to 5.5 by sulphuric acid. 1.2 g activated carbon are added and the solution is stirred for 30 min at 0-5° C. The activated carbon is filtered off.

For crystallisation the filtrate is added dropwise into 280 ml water containing seed crystals of Cefdinir (produced according to US 2003/0204082) and the pH is kept between 2 and 3 by simultaneous addition of diluted sulphuric acid. After completion the crystal suspension is stirred for 1 h at 0-5° C. The white crystals are isolated by filtration and washed 3 times with 60 ml cold water each time.

Drying of the crystals is done in continous nitrogen stream equilibrated to a relative humidity of greater than 58% at 40° C. until the crystals reach an equlibrium relative humidity of 72% at 40° C. (RH measurement as above), to give (6R,7R)-7-[[(2Z)-(2-amino-4-thiazolyl)(acetoxyimino)hydroxy]amino]-3-ethenyl-8-oxo-5-thia-1-azabicyclo[4.2.0]oct-2-ene-2-carboxylic acid crystals (form C), 11.3 g.

### EXAMPLE 3

### Preparation of Form C by Crystallization from Aqueous Solution

11.5 g of cefdinir is dissolved in 450 ml water with addition of NaHCO₃. 1.15 g activated charcoal is added and the mixture is stirred for 15 minutes. The activated charcoal is then filtered off. The obtained filtrate is added at 0-5° C. into water maintaining the pH at 2.0-3.0 by simultaneous addition of dilute sulphuric acid. After completion of addition, the crystal suspension is stirred at 0-5° C. for 30 minutes. The resulting needles are isolated by filtration and dried in a continuous air stream equilibrated to a relative humidity of 52% at 25° C. until the material reaches an equilibrium relative humidity of 58% at 25° C. (RH measurement as above), to give (6R,7R)-7-[[(2Z)-(2-amino-4-thiazolyl)(acetoxyimino)hydroxy]amino]-3-ethenyl-8-oxo-5-thia-1-azabicyclo[4.2.0]oct-2-ene-2-carboxylic acid crystals (form C), 11.0 g.

### EXAMPLE 4

### Preparation of a Dry Suspension Comprising Crystalline Form C of Cefdinir

| | Amount | | |
|---|---|---|---|
| | per | | |
| | unit | | |
| Ingredient: | dose*: | | |
| Cefdinir Monohydrate | 130.8 | mg | (corresponding to 125 mg cefdinir) |
| Sucrose | 2802 | mg | |
| Xanthan gum | 4 | mg | |
| Guar gum | 8 | mg | |
| Citric acid | 3 | mg | |
| Sodium citrate | 3 | mg | |
| Aerosil ® | 10 | mg | |
| Strawberry flavour** | 20 | mg | |

*unit dose per 5 ml of ready made suspension, i.e. dry suspension plus water added in an amount as to obtain 5 ml of suspension
**Strawberry 501094 AP0551 commercially available by Firmenrich; this flavour is also used in Example 5.

Cefdinir monohydrate is spread onto cups or trays and is equilibrated for about 24 hours in air at a relative humidity of 70±5%. Subsequently, the equilibrated product is mixed with part of the sucrose at a relative humidity of 55±10%. The obtained mixture is combined with a mixture of the remaining excipients, and is subsequently mixed. The resulting mixture is then filled into glass bottles having a screw closure at an ambient relative humidity of 55±10%; the filled bottles are subsequently closed tightly by applying an aluminium membrane onto the bottle opening by induction sealing and by applying the screw closure.

### EXAMPLE 5

### Preparation of a Dry Suspension Comprising Crystalline Form C of Cefdinir

| | Amount | | |
|---|---|---|---|
| | per | | |
| | unit | | |
| Ingredient: | dose*: | | |
| Cefdinir Monohydrate | 261.5 | mg | (corresponding to 250 mg cefdinir) |
| Sucrose | 2671 | mg | |
| Xanthan gum | 4 | mg | |
| Guargum | 8 | mg | |
| Citric acid | 3 | mg | |
| Sodium citrate | 3 | mg | |
| Aerosil ® | 20 | mg | |
| Magnesium stearate | 15 | mg | |
| Sodium benzoate | 5 | mg | |
| Strawberry flavour | 30 | mg | |

*unit dose per 5 ml of ready made suspension, i.e. dry suspension plus water added in an amount as to obtain 5 ml of suspension

Cefdinir monohydrate is flushed with air whereof the relative humidity is adjusted to a value of 70±5% in a perforated drum for about 12 hours. Subsequently, the product obtained is mixed with part of the sucrose, with xanthan gum and guar gum at a relative humidity of 55±10%. The following steps are all performed at an ambient relative humidity of 55±10 %: the mixture obtained is then granulated, and the obtained granules are combined with a mixture of the remaining excipients and are mixed. The mixture obtained is then then filled into glass bottles having a screw closure at an ambient relative humidity of 55±10%; the filled bottles are subsequently closed tightly by applying an aluminium membrane onto the bottle opening by induction sealing and by applying the screw closure.

### EXAMPLE 6

### Preparation of a Capsule Comprising Crystalline Form C of Cefdinir

| | Amount | | |
|---|---|---|---|
| | per | | |
| Ingredient: | capsule: | | |
| Cefdinir Monohydrate | 313.8 | mg | (corresponding to 300 mg cefdinir) |
| Magnesium stearate | 5 | mg | |
| Aerosil ® | 1 | mg | |
| Myrj ® 52 | 2 | mg | |
| Calcium-Carmellose | 58 | mg | |

Cefdinir monohydrate is spread onto cups or trays and is equilibrated for about 24 hours in air at a relative humidity of 70±5%. Subsequently, the product obtained is mixed with the mixture of the excipients at a relative humidity of 55±10%. The following steps are all performed at an ambient relative humidity of 55±10%: the obtained mixture is granulated, the obtained granulate is subsequently filled into hard gelatine capsules. The capsules are subsequently filled into polyethylene bottles with a screw closure which are subsequently closed tightly by applying an aluminium membrane onto the bottle opening by induction sealing and by applying the screw closure. The capsules are also packaged or filled into blisters having an Aclar® foil and an aluminium cover foil which are subsequently closed by heat sealing.

### EXAMPLE 7

### Preparation of a Tablet Comprising Crystalline Form C of Cefdinir

| | Amount | | |
|---|---|---|---|
| | per | | |
| Ingredient: | tablet: | | |
| Cefdinir Monohydrate | 261.5 | mg | (corresponding to 250 mg cefdinir) |
| Calcium-Carmellose | 97 | mg | |
| Sodium lauryl sulfate | 3 | mg | |
| Magnesium stearate | 7 | mg | |
| Lactose monohydrate | 48 | mg | |
| Avicel | 20 | mg | |
| Crospovidon | 18 | mg | |

Cefdinir monohydrate is spread onto cups or trays and is equilibrated for about 24 hours in air at a relative humidity of 70±5%. Subsequently, the product obtained is mixed with part of the calcium-carmellose, with part of the magnesium stearate and with sodium lauryl sulfate at a relative humidity of 55±10%. The following steps are all performed at an ambient relative humidity of 55±10%: the mixture obtained is granulated. The granulate obtained is then combined with the remaining parts of calcium-carmellose and magnesium stearate, and with Avicel, Lactose and Crospovidone, and is mixed. The resulting mixture is subsequently compressed to tablets which are subsequently filled into polyethylene bottles with a screw closure which are subsequently closed tightly by applying an aluminium membrane onto the bottle opening by induction sealing and by applying the screw closure. The tablets are also packaged or filled into blisters having an Aclar® foil and an aluminium cover foil which are subsequently closed by heat sealing.

We claim:

1. The compound 7-[2-(2-aminothiazol-4-yl)-2-hydroxyiminoacetamido]-3-vinyl-3-cephem-4-carboxylic acid in the form of a trihydrate.

2. The compound of claim 1 in crystalline form characterized by powder x-ray diffraction peaks at the d-spacings of about 16.68, 8.33, 6.28, 4.17 and 3.24.

3. A process for preparing a crystalline form of 7-[2-(2-aminothiazol-4-yl)-2-hydroxyiminoacetamido]-3-vinyl-3-cephem-4-carboxylic acid according to claim 1 comprising:
a) crystallizing 7-[2-(2-aminothiazol-4-yl)-2-hydroxyiminoacetamido]-3-vinyl-3-cephem-4-carboxylic acid from aqueous solution at a pH adjusted to 1.5 to 4 and at a temperature of −5° C. to 6° C., and
b) drying the crystals at a relative humidity of at least 40%.

4. A pharmaceutical composition comprising crystalline form C of cefdinir wherein the equilibrium relative humidity is at least about 45% as measured by the ERH method.

5. A container comprising a pharmaceutical composition according to claim 4 and a gaseous atmosphere having a relative humidity of at least about 45%.

6. The container of claim 5, wherein the container is capable of maintaining said relative humidity of at least about 45% stable for at least 6 months.

7. A process for preparing a pharmaceutical composition according to claim 4 comprising the steps of
a′) equilibrating a crystalline form of 7-[2-(2-aminothiazol-4-yl)-2-hydroxyiminoacetamido]-3-vinyl-3-cephem-4-carboxylic acid in the form of a hydrate at a relative humidity of at least about 63%;
b′) mixing the equilibrated 7-[2-(2-aminothiazol-4-yl)-2-hydroxyiminoacetamido]-3-vinyl-3-cephem-4-carboxylic acid obtained in step a) with one or more pharmaceutically acceptable excipients at a relative humidity of at least about 45%;
c′) optionally granulating the mixture obtained in step b′) at a relative humidity of at least about 45%; and
d′) further processing the mixture obtained in step b′) or the granulate obtained in step c′) at a relative humidity of at least about 45% to obtain a pharmaceutical composition.

8. The process of claim 7 wherein the further processing of step d′) comprises further mixing the product obtained in step b′) or step c′) with additional pharmaceutically acceptable excipients and optionally compressing the product obtained into tablets.

9. The process of claim 7 wherein the further processing of step d′) comprises filling the granulate obtained in step c′) into capsules.

10. The process of claim 7 comprising the additional step of filling the pharmaceutical composition obtained in step d′) at a relative humidity of at least about 45% into a container capable of maintaining a gaseous atmosphere at a relative humidity of at least about 45% for at least 6 months.

11. The process of claim 8 wherein the further processing of step d′) comprises filling the granulate obtained in step c′) into capsules.

12. The process of claim 8 comprising the additional step of filling the pharmaceutical composition obtained in step d′) at a relative humidity of at least about 45% into a container capable of maintaining a gaseous atmosphere at a relative humidity of at least about 45% for at least 6 months.

13. The process of claim 9 comprising the additional step of filling the pharmaceutical composition obtained in step d′) at a relative humidity of at least about 45% into a container capable of maintaining a gaseous atmosphere at a relative humidity of at least about 45% for at least 6 months.

14. A pharmaceutical composition comprising the compound of claim 1 comprising and optionally, pharmaceutically acceptable excipients.
